(19) United States
(12) Patent Application Publication
(10) Pub. No.: US 2026/0306236 A1
(43) Pub. Date: Oct. 8, 2026

(54) METHODS AND SYSTEMS FOR REMOTE DISTANCE PRINTING

(71) Applicants: MUTHUKUMARAN PACKIRISAMY, PIERREFONDS (CA); MOHSEN HABIBI, MONTREAL (CA); SHERVIN FOROUGHI, MONTREAL (CA)

(72) Inventors: MUTHUKUMARAN PACKIRISAMY, PIERREFONDS (CA); MOHSEN HABIBI, MONTREAL (CA); SHERVIN FOROUGHI, MONTREAL (CA)

(21) Appl. No.: 19/482,547

(22) PCT Filed: May 7, 2024
(86) PCT No.: PCT/CA2024/050618

§ 371 Date: Nov. 7, 2025

Related U.S. Application Data
(60) Provisional application No. 63/500,681, filed on May 8, 2023.

(51) Int. Cl. A61F 2/00; A61B 17/00; A61B 90/00

(52) U.S. Cl. CPC A61F 2/00; A61B 17/00234; A61B 90/37; A61B 2017/00292; A61B 2090/374; A61B 2090/378; A61F 2240/002

(57) ABSTRACT

The present invention introduces a new concept in additive manufacturing (AM) by printing remotely without direct access to printing locations. Common energy sources in AM, heat and light, have small penetration into an optically opaque medium. However, the present invention introduces a deep penetration access to the printing medium using acoustic waves. Acoustic wave patterns create chemically active regions in which the printing material is solidified. The printing material may be a heat curing polymer or polymer composite. This concept introduces a new paradigm in non-invasive/minimally invasive bio-printing inside body, without open surgery, which is proved experimentally in this patent. In addition, a new concept of printing underneath solid and opaque shells with industrial application especially in aerospace is introduced, generated individually with dimensions smaller than those currently achieved;

### CROSS-REFERENCE TO RELATED APPLICATIONS

This patent application claims the benefit of priority from U.S. Patent Application 673/500,681 filed May 8, 2023.

### FIELD OF THE INVENTION

This patent application relates to additive manufacturing and more particularly to acoustically triggered ultra active micro-reactors (UAMRs) to provide remote distance printing of structures and acoustically triggered UAMRs for printing underneath solid and opaque shells with industrial application especially in aerospace or within media such as human or animal body tissue.

### BACKGROUND OF THE INVENTION

Additive manufacturing (AM) processes are generally based on pixel-by-pixel and layer-by-layer solidification of a build material to create three-dimensional objects. Volumetric printing has also recently been introduced to create a three-dimensional image of the desired object in a container filled with printing material. However, despite all of the development on AM within the prior art light and heat are still the dominant energy sources for polymerization or deposition/melting of the printing materials within AM manufacturing processes. These limit the depths of penetration of the energy sources into a medium within which the printing material is solidified.

Accordingly, the inventors have established a new printing paradigm, which they refer to as Remote Distance Printing (RDP), wherein acoustic waves, for example, are employed to induce chemical reactions and drive the solidification process of the printing material, via a sonochemistry route in the scenario of using acoustic waves. Using a triggering mechanism compatible with the triggering of the chemical reactions and signal propagation through intervening media the penetration depth of the triggering mechanism, the energy source, can be flexible and increased dramatically over prior art AM solutions.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to mitigate limitations within the prior art relating to additive manufacturing and more particularly to acoustically triggered ultra active micro-reactors (UAMRs) to provide remote distance printing of structures and acoustically triggered UAMRs for printing underneath solid and opaque shells with industrial application especially in aerospace or within media such as human or animal body tissue.

In accordance with an embodiment of the invention there is provided a method of forming a three dimensional (3D) object comprising:
providing one or more acoustic sources configurable to generate a pressure field comprising at least one of a focused pressure field and an unfocused pressure fields within a print material; and
moving the pressure field by moving at least one of a subset of the one or more acoustic sources or adjusting phases of the pressure fields generated by the one or more acoustic sources; wherein
the pressure field generated by the one or more acoustic sources triggers the generation of a highly chemical active region within the printing material thereby solidifying a portion of the printing material such that the moving pressure field forms the 3D object.

In accordance with an embodiment of the invention there is provided a method of forming a three dimensional (3D) object comprising:
providing one or more energy sources configurable to generate a region triggering a chemical region within a print material; and
moving the region by moving at least one of a subset of the one or more acoustic sources or adjusting phases of the pressure fields generated by the one or more acoustic sources; wherein
the region generated by the one or more energy sources triggers the generation of a highly chemical active region within the printing material thereby solidifying a portion of the printing material such that the moving region forms the 3D object.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIGS. 1A and 1B depict a comparison between light based additive manufacturing (AM) and an embodiment of the invention using an acoustic field in optically opaque materials, respectively;

FIGS. 2A and 2B depict schematically the remote distance printing (RDP) concept and a detailed schematic of an ultra-active micro-reactor (UAMR);

FIG. 3 depicts four different optically opaque polymer composites printed with an RDP system according to an embodiment of the invention;

FIG. 4A depicts sintered opaque printed materials formed using an RDP system according to an embodiment of the invention comprising silica/alumina loaded polydimethylsiloxane (PDMS) (SiO₂/Al₂O₃-PDMS), aluminum loaded PDMS (Al-PDMS) and iron loaded PDMS (Fe-PDMS);

FIG. 4B depicts X-ray diffraction (XRD) patterns for the SiO₂/Al₂O₃-PDMS, Al-PDMS and Fe-PDMS ceramic parts;

FIG. 4C depicts crystalline peak identification for the Al-PDMS ceramic part;

FIG. 4D depicts Fourier transform infra-red (FTIR) spectra for the Fe-PDMS composite after printing (polymeric part) and after pyrolysis (ceramic part);

FIG. 5 depicts a schematic of RDP for deep inside body printing;

FIG. 6A depicts a schematic of an experimental RDP setup employed by the inventors;

FIG. 6B depicts an image of an in-vitro/ex-vivo proof of concept RDP setup;

FIG. 6C depicts a cross-section of a tissue phantom of human skin and muscle employed within prototypes experiments;

FIG. 6D depicts an image of a printed part using the tissue phantom of FIG. 6C;

FIG. 7A depicts a porcine tissue compromising skin, fat and muscle employed within prototype experiments;

FIG. 7B depicts an image of a print part using the porcine tissue of FIG. 7A;

FIGS. 8A and 8B depict 3D models and printed parts for an ear and nose using a tissue phantom; and

FIGS. 9A to 9D depict schematically an RDP concept for printing beneath an opaque shell comprising the steps of printing material injection, filled region formation, printing within desired locations and final solidified piece part.

### DETAILED DESCRIPTION

The present invention is directed to additive manufacturing and more particularly to acoustically triggered ultra active micro-reactors (UAMRs) to provide remote distance printing of structures and acoustically triggered UAMRs for printing underneath solid and opaque shells with industrial application especially in aerospace or within media such as human or animal body tissue.

The ensuing description provides representative embodiment(s) only, and is not intended to limit the scope, applicability, or configuration of the disclosure. Rather, the ensuing description of the embodiment(s) will provide those skilled in the art with an enabling description for implementing an embodiment or embodiments of the invention. It being understood that various changes can be made in the function and arrangement of elements without departing from the spirit and scope as set forth in the appended claims. Accordingly, an embodiment is an example or implementation of the inventions and not the sole implementation. Various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention can also be implemented in a single embodiment or any combination of embodiments.

Reference in the specification to “one embodiment”, “an embodiment”, “some embodiments” or “other embodiments” means that a particular feature, structure, or characteristic described in connection with the embodiments is included in at least one embodiment, but not necessarily all embodiments, of the inventions. The phraseology and terminology employed herein is not to be construed as limiting but is for descriptive purposes only. It is to be understood that where the claims or specification refer to “a” or “an” element, such reference is not to be construed as there being only one of that element. It is to be understood that where the specification states that a component feature, structure, or characteristic “may”, “might”, “can” or “could” be included, that particular component, feature, structure, or characteristic is not required to be included.

Reference to terms such as “left”, “right”, “top”, “bottom”, “front” and “back” are intended for use in respect to the orientation of the particular feature, structure, or element within the figures depicting embodiments of the invention. It would be evident that such directional terminology with respect to the actual use of a device has no specific meaning as the device can be employed in a multiplicity of orientations by the user or users.

Reference to terms “including”, “comprising”, “consisting” and grammatical variants thereof do not preclude the addition of one or more components, features, steps, integers, or groups thereof and that the terms are not to be construed as specifying components, features, steps, or integers. Likewise, the phrase “consisting essentially of”, and grammatical variants thereof, when used herein is not to be construed as excluding additional components, steps, features integers or groups thereof but rather that the additional features, integers, steps, components or groups thereof do not materially alter the basic and novel characteristics of the claimed composition, device or method. If the specification or claims refer to “an additional” element, that does not preclude there being more than one of the additional element.

A “CAD model” as used herein may refer to, but is not limited to, an electronic file containing information relating to a component, piece-part, element, assembly to be manufactured. A CAD model may define an object within a two-dimensional (2D) space or a three-dimensional (3D) space and may in addition to defining the internal and/or external geometry and structure of the object include information relating to the material(s), process(es), dimensions, tolerances, etc. Within embodiments of the invention the CAD model may be generated and transmitted as electronic content to a system providing manufacturing according to one or more embodiments of the invention. Within other embodiments of the invention the CAD model may be derived based upon one or more items of electronic content directly, e.g. a 3D model may be created from a series of 2D images, or extracted from electronic content.

A “fluid” as used herein may refer to, but is not limited to, a substance that continually deforms (flows) under an applied shear stress. Fluids may include, but are not limited to, liquids, gases, plasmas, and some plastic solids.

A “powder” as used herein may refer to, but is not limited to, a dry, bulk solid composed of a large number of exceptionally fine particles that may flow freely when shaken or tilted. Powders may be defined by both a combination of the material or materials they are formed from and the particle dimensions such as minimum, maximum, distribution etc. A powder may typically refer to those granular materials that have fine grain sizes but may also include larger grain sizes depending upon the dimensions of the part being manufactured, the characteristics of the additive manufacturing system etc.

A “metal” as used herein may refer to, but is not limited to, a material having good electrical and thermal conductivity. Metals are generally malleable, fusible, and ductile. Metals as used herein may refer to elements such as gold, silver, copper, aluminum, iron, etc. as well as alloys such as bronze, stainless steel, steel etc.

A “resin” as used herein may refer to, but is not limited to, a solid or highly viscous substance which is typically convertible into polymers. Resins may be plant-derived or synthetic in origin.

A “ceramic” as used herein may refer to, but is not limited to, an inorganic, nonmetallic solid material comprising metal, non-metal or metalloid atoms primarily held in ionic and covalent bonds. Such ceramics may be crystalline materials such as oxide, nitride or carbide materials, elements such as carbon or silicon, and non-crystalline.

A “polymer” as used herein may refer to, but is not limited to, is a large molecule, or macromolecule, composed of many repeated subunits. Such polymers may be natural and synthetic and typically created via polymerization of multiple monomers. Polymers through their large molecular mass may provide unique physical properties, including toughness, viscoelasticity, and a tendency to form glasses and semi-crystalline structures rather than crystals.

An “insulator” as used herein may refer to, but is not limited to, a material whose internal electric charges do not flow freely, and therefore make it nearly impossible to conduct an electric current under the influence of an electric field.

A “robot” or “robotic system” as used herein may refer to, but is not limited to, mechanical systems providing control of movement of a portion or portion of portions of the mechanical system under user or computer control. A robot would have a frame, form or shape designed to achieve a particular task together with electrical components which power and control the robot and some contain some level of computer programming code. A robot may be fixed or mobile and may include a system designed to mimic a biological form, e.g. an android.

An “energy source” as used herein may refer to, but is not limited to, an element creating an emitted signal within an additive manufacturing (AM) system according to or exploiting one or more embodiments of the invention. An energy source may refer solely to that portion of each element generating the emitted signal, e.g. a transducer, or it may refer to the element generating the emitted signal together with part or all of the associated control and drive circuitry receiving control data, processing the control data, and generating the appropriate drive signal(s) to the element generating the emitted signal. An energy source may generate an emitted signal selected from the group comprising infrared (IR) radiation, visible radiation, ultraviolet (UV) radiation, microwave radiation, radio frequency (RF) radiation, X-ray radiation, electron beam radiation, an ultrasonic signal, an acoustic signal, a hypersonic signal, a magnetic field and an electric field. Whilst an energy source may refer to a single emitted signal type other energy sources may emit multiple signals. The physical dimensions of an energy source may vary according to the dimensions of the AM system they form part as well as the number of discretized emitters within the AM system. Accordingly, energy sources may be pico-elements having dimensions defined in picometers (10⁻¹² m) or Angströms (10⁻¹⁰ m), nano-elements having dimensions defined in nanometers (10⁻⁹ m), micro-elements having dimensions defined in micrometers (10⁻⁶ m), as well as elements having dimensions defined in millimeters (10⁻¹² m), centimeters(10⁻² m), meters (10⁰ m) and decameters (10¹ m).

An “X-wave” as used herein may refer to, but is not limited to, a wave or field generated by an energy source which propagates from the energy source through one or more media. An X-wave may accordingly be an emitted wave or field selected from the group comprising near-infrared (IR) radiation, far (IR) radiation, visible radiation, ultraviolet (UV) radiation, microwave radiation, radio frequency (RF) radiation, X-ray radiation, electron beam radiation, an ultrasonic signal, an acoustic signal, a hypersonic signal, a magnetic field and an electric field.

A “nanoparticle” or “ultrafine particle” as used herein may refer to, but is not limited to, a particle of matter that is between 1 and 100 nanometers (nm) in diameter. However, the term may also be employed for larger particles, for example up to 500 nm, or nanofibers (solid fibers with length substantially larger than cross-sectional dimensions) and nanotubes tubes (hollow cored particles with lengths substantially larger than cross-sectional dimensions) that are less than 100 nm in only two directions.

As outlined above additive manufacturing (AM) processes are generally based on pixel-by-pixel and layer-by-layer solidification of the build material to create three-dimensional objects. AM processes are generally viewed as less wasteful than traditional processes based upon subtractive manufacturing (SM), which relate to material removal, as well potential for manufacturing parts at lower cost than those employing SM particularly one-offs and prototypes. However, despite all of this development, light and heat are still the dominant energy sources in AM, for polymerization or deposition/melting of the printing materials. This limits the depth of penetration of the energy sources to the medium in which the printing material is solidified.

In contrast, volumetric printing (VP) has also been recently introduced wherein creation of a three-dimensional image of the desired object in a container filled with printing material is employed, see for example WO/2018/1451947 “Methods and Systems for Additive Manufacturing.” Based upon this the inventors have established a new printing paradigm, which they refer to as Remote Distance Printing (RDP). Within RDP acoustic waves, for example, are employed to induce chemical reactions and drive the solidification process of the printing material, via a sonochemistry route in the scenario of using acoustic waves. By using a triggering mechanism compatible with the triggering of the chemical reactions and transparency of intervening media the penetration depth of the triggering mechanism, the energy source, can be flexible and increased dramatically over prior art AM solutions.

Within the following description acoustic waves are described and employed. However, within the scope of the current invention acoustic waves represent one type of X-wave as used herein to refer to a wave or field generated by an energy source which propagates from the energy source through one or more media to trigger the chemical reaction(s) resulting in formation of the printed object. The printed object may therefore be embedded within one or more media. An X-wave may accordingly be near-infrared (IR) radiation, far (IR) radiation, visible radiation, ultraviolet (UV) radiation, microwave radiation, radio frequency (RF) radiation, X-ray radiation, electron beam radiation, an ultrasonic signal, an acoustic signal, a hypersonic signal, a magnetic field and an electric field. The appropriate X-wave being determined based upon the intervening media being transparent, to a necessary degree such as to either limit undesired absorption by the intervening media or required power of the energy source for example, and the triggering mechanism.

Within the following description the acoustic filed may be, but is not limited to a High Intensity Focused Ultrasound (HIFU) field or a Low Intensity Focused Ultrasound (LIFU) and can have any shape of the acoustic field through the use of one or more energy sources which based upon their geometry, emitted field shape, field amplitude and filed phase relative to others combine to produce the target field. As described within WO/2018/1451947 the shape of this field and the location of the “focused” region where the fields overlap can be varied spatially and temporally so that the focused region can be translated within the media to form the three-dimensional (3D) printed object.

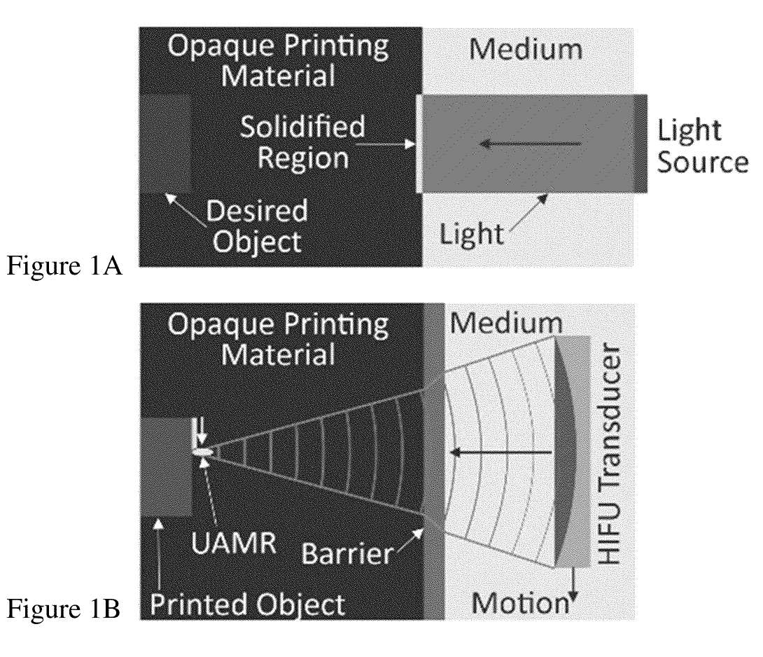

Referring to FIGS. 1A and 1B the difference between a light (photon) based printing technologies and an embodiment of the current invention. Referring to FIG. 1A the depth of penetration of the light in an optically opaque material is limited to sub-microns or sub-millimeter scales. However, with an acoustic field the penetration depth can be millimeters, centimeters or potentially tens of centimeters. Referring to FIG. 1B the acoustic waves drive a sonochemical reaction. The inventors may within this specification refer synonymously to particles that undergo the chemical reaction and a region where the chemical reactions occur as an Ultra Active Micro Reactor (UAMR). In general the term within this specification is applied to the region and the desired 3D part is printed by moving the UAMR within either a medium within which the printing material is present or within the printing material itself. A UAMR may, within some embodiments of the invention, be a small, localized region such that small features of the 3D part or small 3D parts can be formed. A UAMR may, within other embodiments of the invention, a medium/large and extended region. Within embodiments of the invention the UAMR may be created by one or more acoustic holograms and/or metamaterials. A UAMR may be translated by moving the transducer(s) generating the energy source(s) or by using phased array transducer systems such as described within WO/2018/1451947.

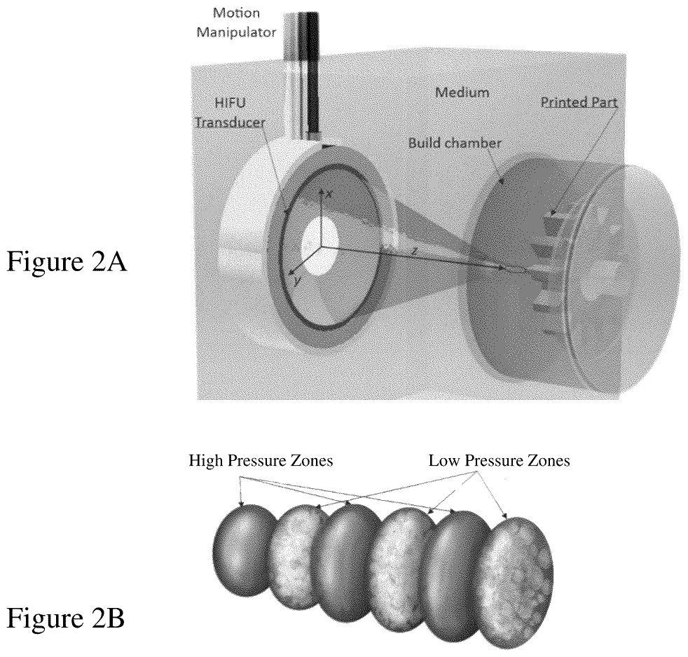

An exemplary printing configuration for RDP according to an embodiment of the invention is depicted in FIG. 2A comprising a HIFU transducer mounted to a motion manipulator where the printing medium is disposed within a build chamber. By appropriate motion of the manipulator and activation of the HIFU transducer the piece part is formed within the medium. FIG. 2B depicts a visualization of a UAMR region where it consists of high and low pressure zones in which the chemically active micro-cavitation “bubbles” are generated driving the solidification process in the region of the UAMR. With such micro-cavitation bubbles heat is generated within the UAMR region thereby curing a heat curing polymer, the printing medium, such as polydimethylsiloxane (PDMS) for example.

In the following description examples of applications of RDP are presented. It would be evident to one of skill in the art that the applications of RDP are not limited to those applications described herein.

### Printing Deep in Opaque Material

Within conventional light-based AM technologies, light absorption and scattering hinder deep penetration of the light (printing energy) into the printing medium as depicted in FIG. 1A especially if the printing material and/or a medium within which the printing material is disposed is opaque and/or filled with scattering particles. However, the optical opacity of the printing material does not affect the RDP process since the sound waves are used as the energy source and not light as shown in FIG. 1B. Acoustic attenuation and scattering that cause absorbance and divergence of the sound waves would be taken into account based on the printing material and/or a medium within which the printing material is disposed when configuring the acoustic signal(s) from the acoustic transducers, e.g. HIFU transducers and/or LIFU transducers. However, due to the nature of sound waves, light absorption and scattering do not affect the depth of sound wave penetration in optically opaque materials as long as acoustic attenuation and scattering are not dominant.

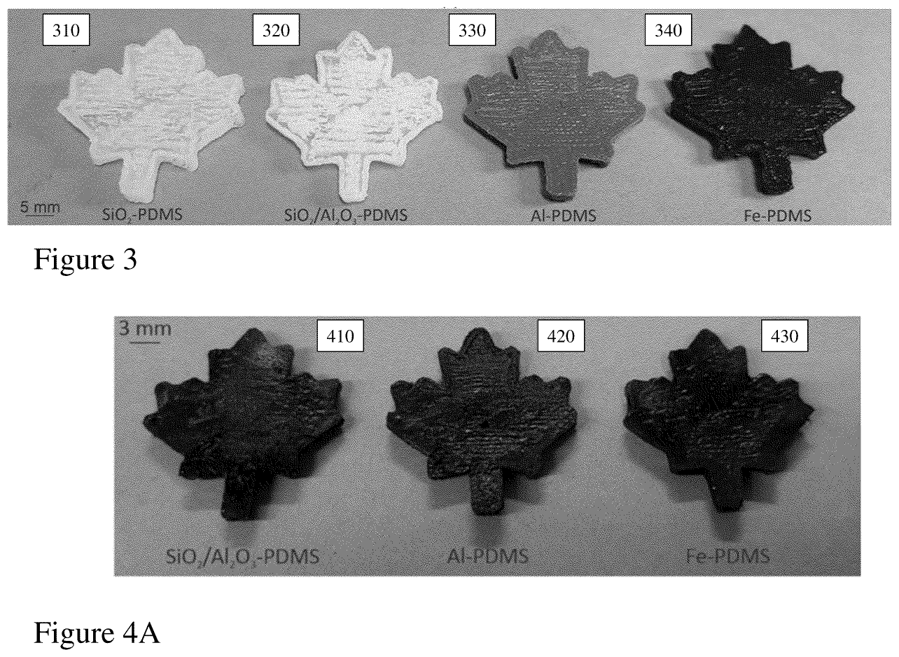

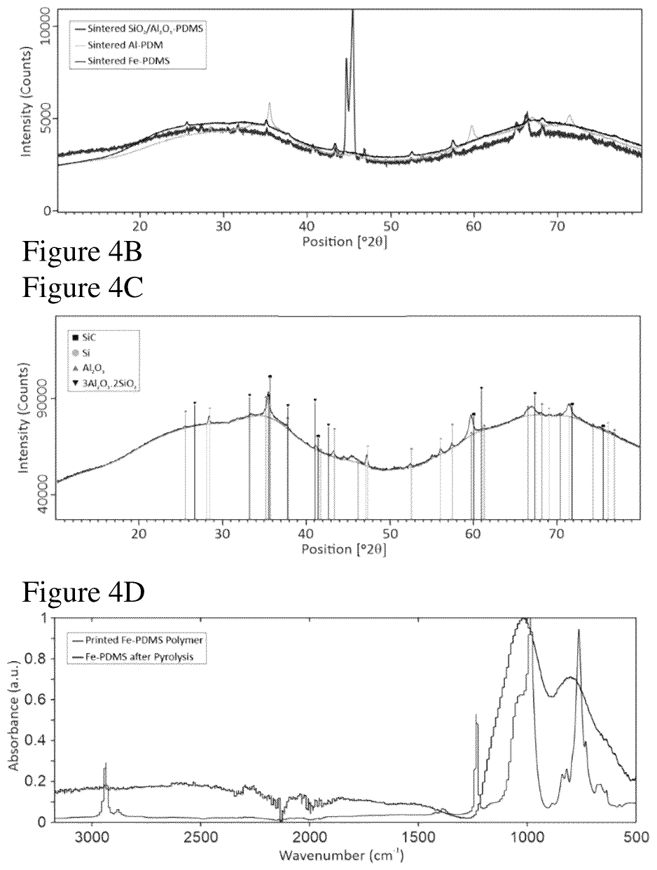

Referring to FIG. 3 there are depicted first to fourth Parts 310 to 340 respectively that are printed from opaque printing materials where the ultrasound waves traveled through 30 mm of water, 1 mm of a solid barrier (wall of the chamber holding the printing material) and 18 mm of the opaque printing material itself to reach the platform upon which the printed parts were formed. The first to fourth Parts 310 to 340 respectively being formed from silica loaded polydimethylsiloxane (PDMS) (SiO₂-PDMS), silica/alumina loaded polydimethylsiloxane (PDMS) (SiO₂/Al₂O₃-PDMS), aluminum loaded PDMS (Al-PDMS) and iron loaded PDMS (Fe-PDMS) respectively. In each instance of printing the opaque micro/nano-composite materials a colloidal solutions of the opaque printing materials was prepared. Accordingly, SiO₂, SiO₂/Al₂O₃, Al and Fe powders were mixed with the PDMS polymer matrix. The printed parts were used as green parts for making ceramic composite objects obtained after sintering in a furnace. First to third Sintered Parts 410 to 430 depict the sintered SiO₂/Al₂O₃-PDMS, Al-PDMS and Fe-PDMS green parts respectively in FIG. 4A.

In order to form the SiO₂ and SiO₂/Al₂O₃ printing materials employed to form the green parts, first and second Parts 310 and 320 respectively as depicted in FIG. 3 7-wt % silica and silica/alumina micro particles respectively were mixed with 84.5-wt % PDMS base-material within a homogenizer for 1 hour before 8.5-wt % curing agent was added and mixed for 15 minutes.

In order to form the printing materials employed to form the green parts, third and fourth Parts 330 and 340 respectively as depicted in FIG. 3 7-wt % of Fe and Al were mixed respectively with 8.5-wt % curing agent and ultrasonically agitated for 10 minutes. The prepared colloidal solution was mixed with 84.5-wt % PDMS base-material for 30 minutes.

These polymer materials respectively were used for preparing the polymer ceramic green parts, first to fourth Parts 310 to 340 respectively, which were then sintered at 1100° C. in a tube furnace under argon gas flow with heating and cooling rates of 40 °C/hour. The resulting SiO₂/Al₂O₃, Al, and Fe sintered ceramic parts being depicted in FIG. 4A.

FIG. 4B depicts X-Ray diffraction (XRD) spectra of these ceramic parts scaled to the background. The measurements were performed upon an Empyrean system using Copper radiation in Bragg-Brentano reflection geometry. The ceramic parts are amorphous with broad peaks with a few weak sharper peaks.

Within FIG. 4C SiC, Si, Al₂O₃ and Al₂O₃·2SiO₂ peaks were identified for the Al-PDMS ceramic part as evident from the peaks in the XRD spectra as evident from FIG. 4C. For the Fe-PDMS sample Fe₃Si, Fe₂ and Fe₅Si₃ were evident with the XRD data.

Fourier-transform infrared spectroscopy (FTIR) of the Fe-PDMS before and after pyrolysis are shown in FIG. 4D. Assignments of infrared (IR) bands in the spectrum of the Fe-PDMS sample (printed part) before pyrolysis were 2950 and 2820 cm−1 (γ(CH₃) and γ(CH₂) stretching vibrations), 1400 cm−1 (δ_{asymm}-Si—CH₃ in-plane symmetrical deformation vibration), 1250 cm−1 (δ_{asymm}-Si—CH₃ in-plane asymmetrical deformation vibration), 1080 and 1015 cm−1 (γ_{Si—O—Si} stretching vibration), 860 cm−1 (Si—CH₃ out-of-plane deformation (rocking) vibration) and 790 cm−1 (γ_{Si—C} stretching vibration). Assignments for the Fe-PDMS after pyrolysis were 1100 and 1080 cm−1 (γ_{Si—O} in Si—O—Si groups) and 770 cm−1 (γ_{Si—C} stretching vibration). The C—H absorption band is visible in the before pyrolysis sample but was not detected after pyrolysis at 2970 cm−1. Both before and after pyrolysis, absorption peaks are visible around 1000 cm−1 where siloxanes have strong bands. No changes in the intensities of the Si—O and Si—C bands, due to the presence of Fe, were observed in the spectrum.

It would accordingly be evident that RDP could have many applications in different disciplines such as remote repairing or on-site maintenance of hidden parts in aerospace industries through to remote in-vivo and noninvasive bio printing of body parts, medical implants etc. medical applications. In the following description exemplary medical applications are outlined.

### Printing Deep Inside Body

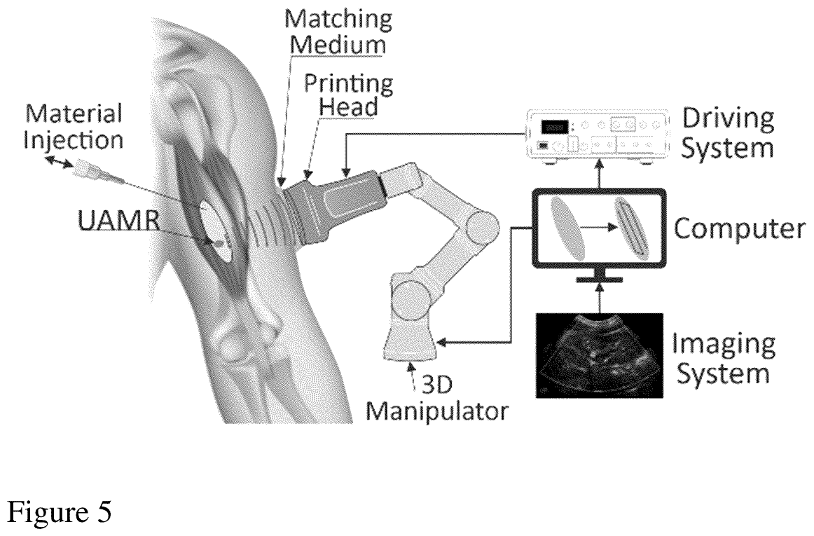

The importance of a combination of printing technologies and injectable biomaterial for the purpose of inside body printing was introduced about 20 years ago, see for example Burg et al. in “Minimally Invasive Tissue Engineering Composites and Cell Printing” (IEEE Eng. Med. Biol. Mag. 22, 84-91, 2003). However, today leading bioprinting technologies require open surgeries due to the fundamental limitations of the current printing methods that use heat or light as the energy source. Recently, near infrared (NIR) sources have been used to print structures noninvasively under skin, 0.5 mm thick. However, this low depth of penetration makes the bioprinting limited to sub-millimeter depths. In contrast, the nature of RDP enables creating structures deep inside body in the range of millimeters to centimeters. A schematic view of an idealized RDP system for noninvasive surgery is depicted in FIG. 5 where printing process is integrated with an imaging system.

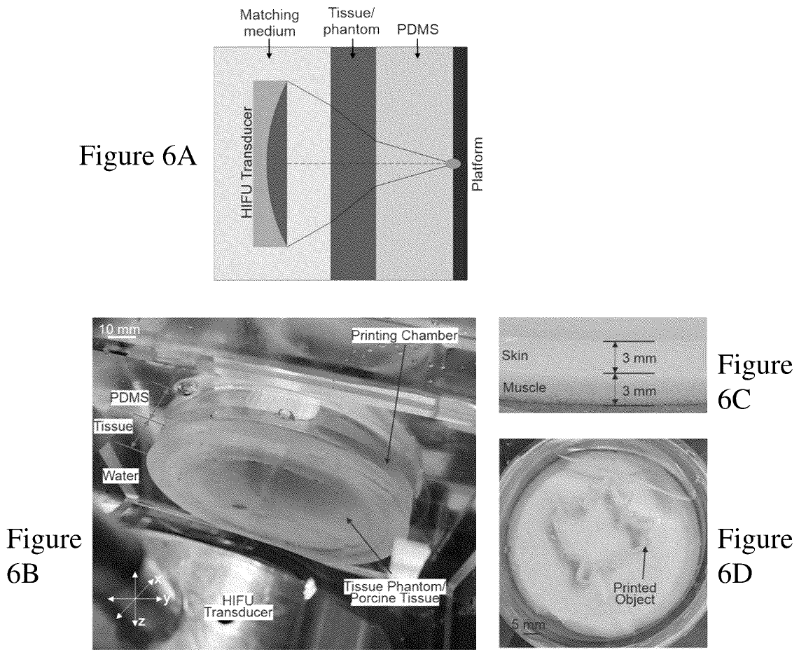

The inventors have demonstrated this concept using an in-vitro/ex-vivo system as depicted in FIG. 6A wherein structures are printed within PDMS with a tissue phantom between the PDMS and a HIFU Transducer disposed within a matching medium. Within the in-vitro experiments performed by the inventors tissue mimicking phantoms of human skin and muscle were created such as depicted in FIG. 6B. The matching medium employed within the experiments was water. The thickness of the tissue phantom being 6 mm. The thickness of the PDMS employed was 18 mm yielding a maximum total printing depth within the PDMS of 24 mm from the water-tissue phantom interface. FIG. 6C depicts a cross-section of the tissue phantom which comprises 3 mm skin and 3 mm tissue. FIG. 6D depicts the printed object attached to the tissue phantom substrate within the printing chamber.

The tissue phantom comprises three layers that mimic the epidermis, dermis and hypodermis layers of real skin tissue. The hypodermis solution was prepared by dissolving 2 % w/v gelatin, 0.2 % w/v agar in 80 ml distilled water along with gradually adding 15 % w/v bovine serum albumin (BSA), and finally mixing with 1 %w/v of silica. The dermis mimicking layer was made from a mixture of 1 % w/v agar, 24 % w/v gelatin, 35 % w/v BSA, and 0.5 % w/v silica microspheres in 80 ml distilled water. The epidermis solution was prepared by mixing 5 % w/v glycerol, 10 % w/v gelatin powder and 0.1 % v/v glutaraldehyde. The 50 ml solution for fabrication of the muscle tissue phantom was made by mixing 40 % v/v evaporated milk, 2 % w/v silica powder, 2 % w/v agar and 60 % v/v distilled water.

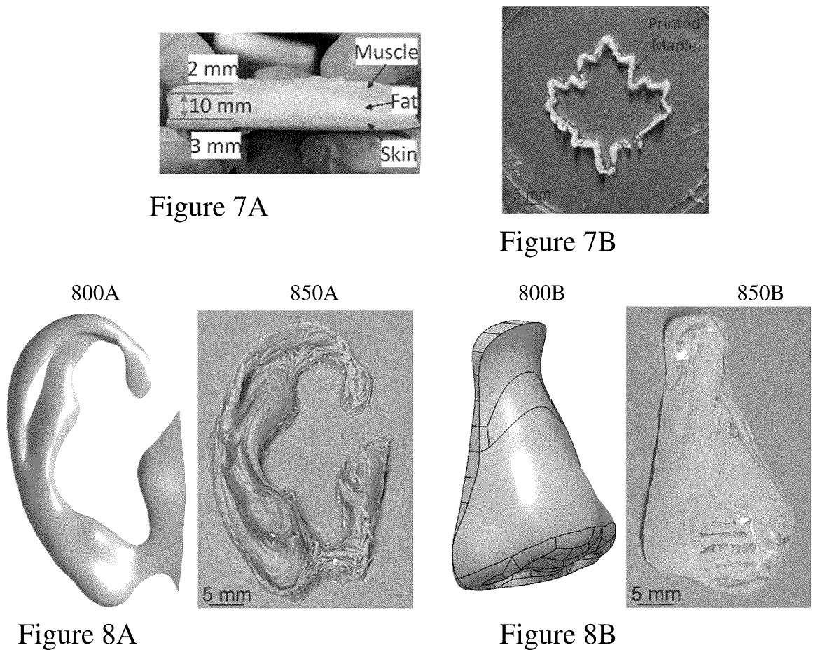

For the ex-vivo experiments the inventors employed porcine tissues such as shown in FIG. 7A comprising 3 mm skin, 10 mm fat and 2 mm muscle. Within these experiments the ultrasound signals pass through 15 mm of tissue (skin, fat and muscle) and up to 18 mm PDMS, i.e. a printing depth of 33 mm. An exemplary printed object being depicted in FIG. 7B.

Referring to FIG. 8A there are depicted a computer aided design (CAD) model 800A of an ear and printed ear 850A. In FIG. 8B there are depicted a CAD design model 800B of a nose and printed nose 850B. The RDP as evident from FIGS. 6A-6D, 7A-7B and 8A-8B allow noninvasive deep inside body printing.

### Printing Deep Underneath a Barrier

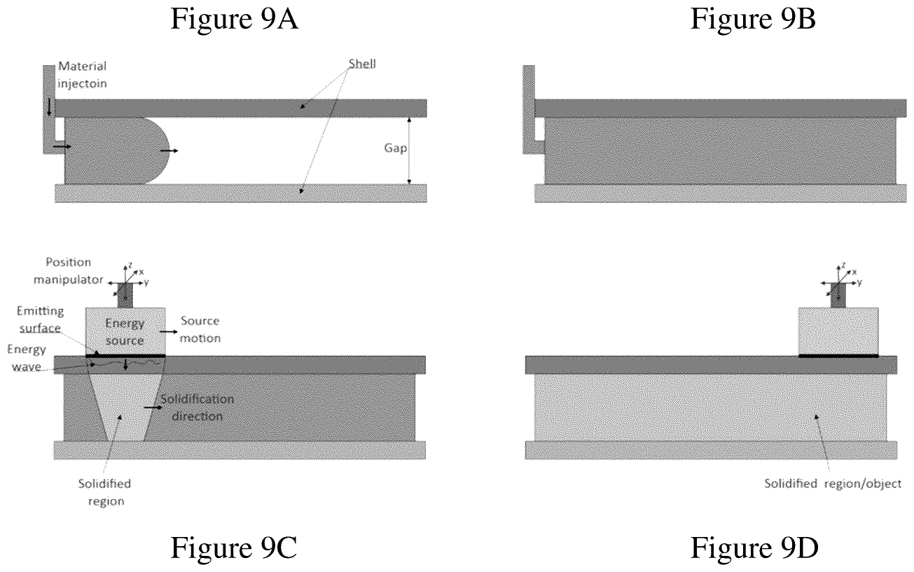

The experimental depictions in FIGS. 6A-8B depict printing under an opaque layer or shell, muscle-tissue-skin-fat. However, within other embodiments of the invention the opaque layer may be a shell formed from metal, plastic, ceramic etc. Referring to FIG. 9A there is depicted a base layer with a shell disposed above it by a gap within which printing material is being injected. The printing material accordingly fills the gap between the shell and base as evident in FIG. 9B. Subsequently, as depicted in FIG. 9C an acoustic source is disposed outside the shell creating the UAMR regions within the printing material which causes solidification of the printing material underneath the shell. Moving the energy source results in multiple chemically active regions creating a series of solidified regions which create the final desired object under the shell as depicted in FIG. 9D.

Within other embodiments of the invention the RDP may print the 3D object behind skin, muscle, fat, and bone for example.

According to an embodiment of the invention at least one of a porosity of a region of the 3D object and a dimension of pores within a porous region of the 3D object are adjusted in dependence upon one or more properties of the printing material.

According to an embodiment of the invention at least one of a porosity of a region of the 3D object, a dimension of the pores within the region and whether the pores are interconnected or disconnected is adjusted in dependence upon an external pressure applied to the printing material.

According to an embodiment of the invention at least one of a porosity of a region of the 3D object, a dimension of the pores within the region and whether the pores are interconnected or disconnected is adjusted in dependence upon at least one of a velocity and an acceleration of motion of the one or more energy sources during formation of the region.

According to an embodiment of the invention the printing material is a resin selected from a liquid resin, a composite resin and a resin slurry wherein a resin matrix of the resin is polymerized via at least one of a free radical polymerization mechanism and a heat cutting process.

According to an embodiment of the invention a pressure field within the printing material is adjusted by moving the pressure field by adjusting phases of the pressure fields generated by the one or more acoustic sources comprises adjusting a characteristic of one or more phased array transducers.

According to an embodiment of the invention a pressure field within the printing material is adjusted by moving the pressure field by adjusting phases of the pressure fields generated by the one or more acoustic sources comprises adjusting a characteristic of at least one of an active acoustic hologram and a metamaterials in real time during the printing process.

According to an embodiment of the invention the printing material is transported to region of a body by a fluid within the body, e.g. saliva, blood, etc. wherein the printing material generates the printed object at a region within the body by that region of the body being exposed to the energy source. The energy source may be selected from emitted wave or field selected from the group comprising microwave radiation, radio frequency (RF) radiation, X-ray radiation, electron beam radiation, an ultrasonic signal, an acoustic signal, a hypersonic signal, a magnetic field and an electric field. The printing material may be added to the fluid, pass through one or more regions of the body and then be removed, e.g., filtered out. For example, a stent may be directly formed within an artery or vein. Within another example a coating may be formed upon a surface of an artery, a vein, bone etc.

According to an embodiment of the invention the printing material is at least one of a heat curing polymer, a biocompatible resin, a heat curing polymer composite and a heat curing bio-compatible ink loaded disposed within a carrier. Within embodiments of the invention the carrier comprises at least one or more types of living cell.

According to embodiments of the invention there is provided a method for forming a three-dimensional (3D) object within a target body by inserting one or more energy sources within the target body wherein the one or more energy sources trigger a series of UAMR regions (activation region) within a printing medium disposed within the target body to form the 3D object at least one of within the target body and a surface of the target body. The one or more energy sources can be moved to move the activation region within the target body.

For example, the target body may be a region of a human body or an animal body such as an artery, a vein, a mouth, an ear, a nose, a throat, a lung, a vagina, a heart, a capillary, and a urinary tract for example. The one or more energy sources may be mounted upon or form part of a flexible device or rigid device moved under the direction of at least one of a user and a robotic system. A flexible device may include, but not be limited to, a catheter or an endoscope. The flexible device or rigid device may include one or more fluidic channels for dispensing the printing medium in the vicinity of the one or more energy sources. The flexible device or rigid device may include one or more other fluidic channels for removing one or more substances and/or byproducts of the UAMRs. The

The 3D fabrication process can be controlled in a closed loop manner with external and/or internal observatory systems such as endoscopy, ultrasound imaging, and magnetic resonance imaging for example.

Within embodiments of the invention the 3D fabrication processes can be performed to fabricate in situ within living tissue one or more of a body part, a portion of a body part, a medical implant, an addition to a medical implant, repair a medical implant, a biocompatible construct, an electrical circuit, muscle, cartilage, body tissue, an under skin tattoo.

Within embodiments of the invention the UAMRs may be employed to trigger one or more subsequent processes that result in removal of a substance from a surface of or within living tissue including, but not limited to, removal of a tattoo, removal of plaque, and treatment of atherosclerosis.

Within embodiments of the invention a printing material after activation through UAMRs may be form a resin, a plastic, a metal, an alloy, a ceramic, a composite, a biocompatible composite, an electrically conductive material, an insulator, a magnetic material, a material with fluorescent properties, and a biomaterial supporting detection of one or more properties including, but not limited to, glucose and a hormone.

It would be evident to one of skill in the art that the methods described and presented may be employed within a wide range of applications including medicine, veterinary medicine, aerospace, transportation systems or vehicles, etc.

Specific details are given in the above description to provide a thorough understanding of the embodiments. However, it is understood that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Also, it is noted that the embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be rearranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A method for forming a three dimensional (3D) object comprising:
providing one or more acoustic sources configurable to generate a pressure field comprising at least one of a focused pressure field and an unfocused pressure fields within a print material; and
moving the pressure field by moving at least one of a subset of the one or more acoustic sources or adjusting phases of the pressure fields generated by the one or more acoustic sources; wherein
the pressure field generated by the one or more acoustic sources triggers the generation of a highly chemical active region within the printing material thereby solidifying a portion of the printing material such that the moving pressure field forms the 3D object.

2. The method according to claim 1, wherein
the 3D object formed within the printed material comprises at least one of a porous portion and non-porous portion.

3. The method according to claim 1, wherein
the printing material is opaque; and
the 3D object is printed deep within the printing material.

4. The method according to claim 1, wherein
at least one of:
the printing material is at least one of a heat curing polymer, a biocompatible resin, a heat curing polymer composite and a heat curing biocompatible ink loaded disposed within a carrier; and
the printing material is at least one of a heat curing polymer, a biocompatible resin, a heat curing polymer composite and a heat curing biocompatible ink loaded disposed within a carrier comprising at least one or more types of living cell.

5. The method according to claim 1, wherein
at least one bone, skin, fat and muscle of at least one of a human and an animal is disposed between the printing material and the one or more acoustic sources.

6. The method according to claim 1, wherein
a shell is disposed between the printing material and the one or more acoustic sources and the printing material cannot be directly accessed.

7. The method according to claim 1, wherein
at least one of a porosity of a region of the 3D object and a dimension of pores within a porous region of the 3D object are adjusted in dependence upon one or more properties of the printing material.

8. The method according to claim 1, wherein
at least one of a porosity of a region of the 3D object, a dimension of the pores within the region and whether the pores are interconnected or disconnected is adjusted in dependence upon an external pressure applied to the printing material.

9. The method according to claim 1, wherein
at least one of a porosity of a region of the 3D object, a dimension of the pores within the region and whether the pores are interconnected or disconnected is adjusted in dependence upon at least one of a velocity and an acceleration of motion of the one or more energy sources during formation of the region.

10. The method according to claim 1, wherein
the printing material is a resin selected from a liquid resin, a composite resin and a resin slurry wherein a resin matrix of the resin is polymerized via at least one of a free radical polymerization mechanism and a heat cutting process.

11. The method according to claim 1, wherein
moving the pressure field by adjusting phases of the pressure fields generated by the one or more acoustic sources comprises adjusting a characteristic of one or more phased array transducers.

12. The method according to claim 1, wherein
moving the pressure field by adjusting phases of the pressure fields generated by the one or more acoustic sources comprises adjusting a characteristic of at least one of an active acoustic hologram and a metamaterials in real time during the printing process.

13. A method for forming a three dimensional (3D) object comprising:
providing one or more energy sources configurable to generate a region triggering a chemical region within a print material; and
moving the region by moving at least one of a subset of the one or more acoustic sources or adjusting phases of the pressure fields generated by the one or more acoustic sources; wherein
the region generated by the one or more energy sources triggers the generation of a highly chemical active region within the printing material thereby solidifying a portion of the printing material such that the moving region forms the 3D object.

14. The method according to claim 13, wherein
each energy source of the one or more energy sources is a source of microwave radiation, radio frequency (RF) radiation, X-ray radiation, electron beam radiation, an ultrasonic signal, an acoustic signal, a hypersonic signal, a magnetic field and an electric field.

15. A method for forming a three-dimensional (3D) object within a target body comprising:
inserting one or more energy sources within the target body where the one or more energy sources trigger a series of ultra active micro-reactor (UAMR) regions (activation regions) within a printing medium disposed within the target body; wherein
the 3D object is at least one of within the target body and upon a surface of the target body; and
the one or more energy sources can be moved to move the activation region within the target body.

16. The method according to claim 15, wherein
the target body is one of a region of a human body and a region of an animal body.

17. The method according to claim 15, wherein
the target body is one of an artery, a vein, a mouth, an ear, a nose, a throat, a lung, a vagina, a heart, a capillary, and a urinary tract.

18. The method according to claim 15, wherein
the one or more energy sources are mounted upon or form part of a flexible device or rigid device; and
the flexible device or rigid device is moved under the direction of at least one of a user and a robotic system.

19. The method according to claim 15, wherein
the one or more energy sources are mounted upon or form part of a catheter or an endoscope.

20. The method according to claim 19, wherein
the flexible device or rigid device includes at least one of:
one or more fluidic channels for dispensing the printing medium in the vicinity of the one or more energy sources; and
one or more other fluidic channels for removing at least one of a substance and a byproducts of the series of UAMRs.

21. The method according to claim 15, wherein
the one or more energy sources are moved in dependence upon a control loop employing at least one of an external observatory system and an internal observatory system.

22. The method according to claim 21, wherein
when the control loop employs an internal observatory system it is endoscopy; and
when the control loop employs an external observatory system it is ultrasound imaging or magnetic resonance imaging.

23. The method according to claim 15, wherein
the formation of the 3D object is performed in situ within living tissue; and
the 3D object is at least one of a body part, a portion of a body part, a medical implant, an addition to a medical implant, repair a medical implant, a biocompatible construct, an electrical circuit, muscle, cartilage, body tissue, an under skin tattoo.

24. The method according to claim 15, wherein
the series of UAMRs trigger one or more subsequent processes that result in removal of a substance from a surface of or within living tissue.

25. The method according to claim 15, wherein
the printing material after activation through the series of UAMRs is one of a resin, a plastic, a metal, an alloy, a ceramic, a composite, a biocompatible composite, an electrically conductive material, an insulator, a magnetic material, a material with fluorescent properties, and a biomaterial supporting detection of one or more properties.
